(19) United States
(12) Patent Application Publication
(10) Pub. No.: US 2015/0132826 A1
(43) Pub. Date: May 14, 2015

(54) CHIMERIC TRUNCATED TISSUE PLASMINOGEN ACTIVATOR (t-PA) RESIATANT TO PLASMINOGEN ACTIVATOR INHIBITOR-1 AND IMPROVED BIOCHEMICAL PROPERTIES

(71) Applicant: Pasteur Institute of Iran (IPI), Tehran (IR)

(72) Inventors: Amirhossein Saadatirad, Tehran (IR); Fereidoun Mahboudi, Tehran (IR); Soroush Sardari, Tehran (IR); Mohammadreza Kazemali, Tehran (IR); Fatemeh Davami, Tehran (IR); Keyvan Majidzadeh, TEHRAN (IR)

(21) Appl. No.: 14/594,155

(22) Filed: Jan. 11, 2015

(51) Int. Cl. C12N 9/72

(52) U.S. Cl. CPC C12N 9/6459
USPC 435/226

(57) ABSTRACT

The present invention discloses a thrombolytic therapy for acute myocardial infarction by t-PA. A chimeric truncated form of t-PA is designed and expressed in Pichia pastoris. The new variant t-PA comprises of a finger domain of Desmoteplase, an epidermal growth factor (EGF) domain, a kringle 1 domain, a kringle 2 domain in which the lysine binging site is deleted, and a protease domain where the four amino acids lysine 296, arginine 298, arginine 299, and arginine 304 are substituted by aspartic acid. The chimeric t-PA shows has increased activity of 14 fold in presence of fibrin. The t-PA shows 10-fold increased potency than commercially available full length t-PA (Actylase®) and provides 1.2 fold greater affinity to fibrin. Further a residual activity of only 68% is observed after incubation of Actylase® with PAI-1 and 91% residual activity for t-PA. The t-PA variant is acceptable plasminogen activator with enhanced biochemical properties.

### CROSS REFERENCE TO RELATED APPLICATION

The present application claims the benefit and the priority of the U.S. Provisional Patent application Ser. No. 62/051,708 filed on Sep. 17, 2014 with title, “Expression of a Novel Chimeric-Truncated tPA in Pichia pastoris with improved Biochemical Properties”, and the contents of which is incorporated in its entirety as reference herein.

### BACKGROUND

1. Technical Field

The embodiments herein generally relate to the field of bio-engineering of drugs by recombinant technology. The embodiment herein particularly relate to the synthesis of thrombolytic drugs and particularly to tissue plasminogen activators (t-PA). The embodiments herein more particularly relate to a novel variant of tissue plasminogen activator (t-PA) with improved pharmacodynamic properties compared to native tissue plasminogen activator.

2. Description of the Related Art

Coronary heart disease (CHD) is the most common form of heart and cardiovascular diseases. Acute ischemic stroke (AIS) is the most common cause of death which is classified into different categories according to the presumed mechanism. Cardioembolic strokes present a major form of ischemic strokes and acute myocardial infarction (AMI). The acute myocardial infarction (AMI) is a cardiac condition that has been associated with cardioembolic strokes. AMI is commonly caused by atherosclerotic occlusion of the coronary arteries, which is the result of a thrombus or clot forming on top of a ruptured atherosclerotic plaque, thereby blocking the blood flow through the artery.

Recognition of the importance of fibrinolytic system in thrombus resolution has resulted in the development of various fibrinolytic agents and plasminogen activators (PAs) with different pharmacokinetic and pharmacodynamic properties.

Plasminogen activators are of great clinical significance as thrombolytic agents for management of stroke and myocardial infarction. Tissue-type plasminogen activator (t-PA) is generally preferred for its more efficacy and safety compared to urokinase and streptokinase. Tissue-type plasminogen activator (t-PA) is a glycoprotein consisting of 527 amino acid residues (72 KDa) with seventeen disulfide bonds and approximate 7% carbohydrate of total molecular weight. The t-PA has enhanced activity in the presence of fibrin, i.e. fibrin-specific plasminogen activation is the major advantage of t-PA over other thrombolytic agents. The tissue-type plasminogen activator (t-PA) is mainly released by endothelial cells. The t-PA cleaves the zymogen plasminogen into active plasmin. Further the plasmin degrades fibrin, which is major component of blood clots, and promotes blood reperfusion. The type-1 plasminogen-activator inhibitor (PAI-1) and a2-antiplasmin (a2-AP) inhibit this cascade by blocking the proteolytic activity of t-PA and plasmin, respectively. The PAI-1 belongs to serpin family which plays its role as an ideal pseudo-substrate for target serine proteases. The first source of PAI-1 is synthesized by endothelial cells and/or by hepatocytes. The second pool of PAI-1 is contained within the a-granules of platelets. The interaction between t-PA and PAI-1 bound to fibrin is composed of three sequential steps: (a) an interaction of the catalytic site of t-PA with the reactive center of PAI-1, bound to fibrin, (b) a conformational change in the t-PA and PAI-1 complex that leads to loss of its affinity for fibrin, and (c) the dissociation of the t-PA and PAI-1 complex from the fibrin matrix and rebinding to fibrin subsequently; that would greatly impede t-PA activity.

Tissue-type plasminogen activator (t-PA) is the dominant t-PA involved in fibrinolysis. The t-PA is a glycoprotein with 67 kDa, 527 amino acids, which promotes conversion of plasminogen to plasmin in the presence of fibrin. The protein molecule is divided into five structural domains: finger domain (F) followed by a growth factor domain (EGF) near the N-terminal region and the two kringle 1 (K1) and kringle 2 (K2) domains. Next to kringle 2 domain is the serine protease domain with the catalytic site at the C terminus. Both finger and kringle 2 bind to the fibrin and accelerate t-PA activation on plasminogen. However, full length t-PA has some major disadvantages i.e. the rapid clearance from plasma due to the recognition of structural elements on first three N-terminal domains by certain hepatic receptors is the most important. Human fibrinogen is converted to fibrin through thrombin catalysis and release of small peptides from the amino-terminal segments of the K and L chains that are named fibrino-peptides A and B, respectively. The tetrapeptide GHRP interacts with a complementary site on the L lobe of fibrin monomers and prevent polymerization. Furthermore, it has been reported that histidine-16 of the BL chain plays an important role in the association of fibrin.

Three different generations of plasminogen activators (PAs) have been introduced to the market. The first generation agents are Streptokinase and Urokinase. The second generation agents are Alteplase® and Acylated plasminogen streptokinase activator complex (APSAC). The third generation agents are Vampire bat plasminogen activator (BatPA), Reteplase®, Tenecteplase®, Lanatoplase®, and Staphylokinase®.

The limited fibrin specificity of t-PA has prompted the development of plasminogen activators (PAs) with greater selectivity for fibrin. Thrombolytic therapy has been shown to significantly improve survival following AMI. The most common thrombolytic agents are Alteplase® (tissue-type plasminogen activator, t-PA) Reteplase®, Tenecteplase®, and Lantoplase®.

Despite all progress made, current thrombolytic therapy is still associated with significant drawbacks including the need for large therapeutic doses, short half-life of the agent due to interaction with plasminogen activator inhibitor-1 (PAI-1), limited fibrin specificity and the risk of either severe bleeding complications or reocclusion.

Resistance to PAI-1 is another factor which confers clinical benefits in thrombolytic therapy. The only US FDA approved PAI-1 resistant drug is Tenecteplase®. Deletion variants of t-PA have the advantage of fewer disulfide bonds in addition to higher plasma half lives.

Development of various forms of t-PA (e.g. Alteplase®, Reteplase® and Tenecteplase®) has exploited the activity of t-PA. Since the recognition that residues 296-304 are critical for the interaction of t-PA with PAI-1, several variants oft-PA with mutations or deletions in this domain have been investigated. Tenecteplase® is the only FDA approved PAI-1 resistant thrombolytic agent. Tenecteplase® consists of two point mutations at positions 103, 117 that causes prolonged plasma half life. Furthermore, the four amino acids at position 296-299 have been replaced by four alanines which provides resistance against the inhibition by PAI-1. Reteplase® is a single-chain non-glycosylated deletion variant of t-PA consisting of only the second kringle and the protease domains. Since finger domain is the responsible domain for fibrin affinity, Reteplase® is characterized by reduced fibrin selectivity and causes more fibrinogen depletion than the full length forms. In the absence of fibrin, Reteplase and Alteplase do not differ with respect to their activity as plasminogen activators, nor do they differ in terms of their inhibition by the PAI-1.

Hence there is a need to develop a variant of tissue plasminogen activator (t-PA) that has more fibrin activity. Also there is a need to develop a variant of tissue plasminogen activator (t-PA) resistant to plasminogen activator inhibitor-1 (PAI-1).

The above mentioned shortcomings, disadvantages and problems are addressed herein and which will be understood by reading and studying the following specification.

### OBJECTIVES OF THE EMBODIMENTS

The primary objective of the embodiment herein is to provide a variant of tissue plasminogen activator (t-PA) which has more serine protease activity in presence of fibrin.

Another object of the embodiment herein is to provide a novel variant of tissue plasminogen activator which has greater fibrin binding compared to the wild tPA.

Yet another object of the embodiment herein is to provide a novel mutant variant of tissue plasminogen activator which is resistant to plasminogen activator-1 (PAI-1).

Yet another object of the embodiment herein is to provide a mutant variant of tissue plasminogen activator which does not cause much depletion of fibrinogen compared to the wild tPA.

Yet another object of the embodiment herein is to provide a mutant variant of tissue plasminogen activator having a fibrin affinity of 1.5 fold compared to native full lengths tPA.

Yet another object of the embodiment herein is to express the mutant variant of tissue plasminogen activator in the Pichia pastoris.

The embodiment herein will become readily apparent from the following detailed description taken in conjunction with the accompanying drawings.

### SUMMARY

The various embodiments herein provide a novel chimeric truncated form of tissue plasminogen activator (t-PA). The chimeric truncated form of tissue plasminogen activator (t-PA) is expressed in Pichia pastoris. The novel tissue plasminogen activator (t-PA) comprises of a finger domain of Desmoteplase, an epidermal growth factor (EGF) domain, a kringle 1 (K1) domain, a kringle 2 (K2) domain and a protease domain. In kringle 2 (K2) domain the lysine binding site (LBS) is deleted. In protease domain the four amino acids lysine 296, arginine 298, arginine 299 and arginine 304 are substituted by aspartic acid (DDDD). The chimeric truncated form of tissue plasminogen activator (t-PA) show increased activity in the presence of fibrin. Further chimeric truncated form of tissue plasminogen activator (t-PA) shows greater affinity towards fibrin than commercially available full length t-PA (Actylase®).

According to one embodiment herein, a chimeric truncated tissue plasminogen activator CT t-PA comprises of a native human t-PA with a F domain, an EGF domain, a K1 domain, a K2 domain and a protease (P) domain. The F domain of native human t-PA is replaced by F domain of vampire bat plasminogen activator. The 24 amino acids (LBS) of the K2 domain are deleted at a position of 202-225. The amino acids K296, R298, R299 and R304 in the P domain are replaced by four aspartic acids (DDDD).

According to one embodiment herein, the chimeric truncated t-PA has 503 amino acids. The chimeric truncated t-PA has a molecular weight of 65 kDa. The chimeric truncated t-PA has a 3N-glycosylation at residues N117, N184 and N448. The chimeric truncated t-PA has a specific activity of 5,200 IU/μg. The chimeric truncated tPA is resistance to a plasminogen activator inhibitor-1 (PAI-1) and the CT t-PA retains more than 90% of its biological activity in the presence of a fibrinogen. The chimeric truncated t-PA has a residual activity of 91%. Also the chimeric truncated t-PA has an amodolytic activity of 1,860 IU/ml in the absence of fibrin. The chimeric truncated t-PA has an amidolytic activity of 2,500,000 IU/ml in the presence of fibrin.

According to one embodiment herein, the method of synthesizing chimeric truncated form of tissue plasminogen activator (t-PA) comprises selecting the Escherichia coli strain which is recombination deficient and deficient in endonuclease A. The Pichia pastorisis is utilized for the expression of chimeric truncated form of tissue plasminogen activator (t-PA).

According to one embodiment herein, after selecting the microbial strains, the gene of interest is designed. To increase the fibrin affinity and specificity the vampire bat plasminogen activator (bPA) is replaced by the human t-PA finger domain and 24 amino acids in the K2 domain known as LBS are deleted. For half-life prolongation, the amino acids K296, R298, R299, and R304 are replaced by four aspartic acids (D) in the protease domain, responsible for the resistance to the PAI-1. The nucleotide sequence of the novel CT t-PA gene encodes a protein of 503 amino acids with a molecular weight of 65 kDa and has 3 N-glycosylation sites at residues N117, N184, and N448. The CT t-PA comprises a Desmoteplase finger domain (f(vamp)) followed by a full length human t-PA EGF domain and K1 domain. Downstream of these domains are the human t-PA K2 domain with the LBS deletion (D 202-225) and the protease domain with four aspartic acids substitutions (DDDD) at residues 296, 298, 299, and 304.

According to one embodiment herein, the expression plasmid pPICZaA/CT t-PA is constructed. The gene coding for the new CT t-PA is synthesized in pGH-30230 plasmid and has an ampicillin selected marker as well as Xho1 and Xba1 restriction sites flanking the gene. The vector for the production of CT t-PA in Pichia pastorisis is constructed using the pPICZaA vector as backbone. The final vector provided the alpha mating factor from Saccharomyces cerevisiae at the 5′ end of the target gene to allow secretion as well a His₆-tag at the 3′ end for simple downstream process. The plasmid (pPICZaA/CT t-PA) is transformed into Escherichia coli and selected on LB plates containing 25 μg/ml Zeocin™. Transformants are selected and verified by PCR, sequencing and digestion analysis. One positive transformant is grown in 100 ml liquid LB comprising Zeocin™ (25 μg/ml) for 12 hours and the recombinant plasmid (pPICZaA/CT t-PA) is isolated using a QIA quick column and sequenced.

According to one embodiment herein, the next step comprises transformation, selection and analysis of Pichia pastorisis clones. 10-20 μg of the recombinant plasmid are linearized using Sac1 and are transformed into Pichia pastorisis GS 115 by electroporation using a MicroPulser. The parent construct pPICZaA which lacks an insert is used as negative control. After transformation the cells are spread on YPD plates comprising Zeocin™ (0.1 mg/ml) and incubated at 30° C. for 3 days. Large colonies are selected and the integration of the CT t-PA gene into the genome is confiremed by direct colony PCR using 5′ AOX1 and 3′ AOX1 primers. The PCR reactions comprises 5 μl of 10×PCR buffer, 2.5 μl of 50 mM MgCl₂, 2.5 μl of 50 mM MgCl₂, 2.5 μl of 10 mM dNTP, and 50 pmol of each primer (final volume 25 μl). The PCR reaction is carried out for 30 cycles. The PCR reaction is incubated 95° C. for 5 min before adding the 2 units of Taq DNA polymerase. Each cycle consists of 1 min at 95° C., 30 sec at 65° C. and 1 min at 72° C. with a final extension step of 5 min at 72° C.

According to one embodiment herein, the Pichia pastorisis clones are subjected to the determination of methanol utilization (Mut) phenotype of clones. The Pichia pastorisis clones are further subjected to high concentration of Zeocin™ to check the resistant.

According to one embodiment herein, the chimeric truncated tissue plasminogen activator (t-PA) production is done by subjecting the transformed Pichia pastorisis to a shake flask. The glycerol stocks are refreshed on YPD agar plates comprising 0.1 mg/ml Zeocin™. Then the transformed Pichia pastorisis cells are cultivated in 100 ml fresh BMGY medium in a 250 ml baffled flask at 30° C. and 250 rpm over night. The culture is harvested by centrifuging the medium at 1500 g at room temperature for 5 min. The pellet is collected and re-suspended in 200 ml of BMMY in a 1 liter baffled flask. The induction is performed for 5 days at 25° C. at 250 rpm. Methanol (1% v/v) is added every 24 hour to maintain induction. The samples are taken at 24 hour interval. The samples are centrifuged and the clarified supernatants are diluted 20-fold in phosphate buffer saline (PBS). The diluted supernatant is subjected for further analysis.

According to one embodiment herein, the chimeric truncated tissue plasminogen activator t-PA protein is purified from the culture medium with fast protein liquid chromatography (FPLC) using Ni-NTA agarose. Approximately a 15 ml Ni-NTA gel bed in a XK 16/20 column is equilibrated with 10 column volumes of 50 mM sodium phosphate pH 8.0, 300 mM NaCl, 10 mM imidazole. Cell free cultivation broth is dialyzed in PBS buffer using a 25 kDa cut off Spectra/Por® membrane tube at 4° C. over night. Then the sample is loaded onto the Ni-NTA resin. Bound proteins are washed using 50 mM sodium phosphate pH 8.0, 0.3 M NaCl and 20 mM imidazole (5CV) and eluted with 50 mM sodium phosphate pH 8.0, 0.3 M NaCl, 300 mM imidazole into eight fractions which are then analyzed by SDS-PAGE.

According to one embodiment herein, the total protein estimation is done by Bradford method. The bovine serum albumin (BSA) is used as standard in concentration of 0.1-1.0 mg/ml.

According to one embodiment herein, the SDS-PAGE and western blotting are carried out using a 12% resolving polyacrylamide gel and stained with coomassie blue R-250. The standard protocol of the kit is followed.

According to one embodiment herein, the activity test of the chimeric truncated tissue plasminogen activator t-PA protein is performed using the Trinilize t-PA Activity kit which is a bio-functional immunosorbent assay (BIA) to quantify the activity of human t-PA. 100 μl of t-PA standards and culture broth supernatant and culture broth supernatant are added to the microtest strip wells and are incubated on a microtest plate shaker at ambient temperature (18-25° C.) at 600 rpm for 20 min. The samples are captured by SP-322 monoclonal antibody on the microtest wells (pH 5.9) without inhibiting t-PA activity. After discarding the test samples, mild detergents are used to wash the wells. The t-PA substrates (plasminogen, a plasmin sensitive chromogenic substrate and t-PA activity promoters) are added in HEPES buffer (pH 8.5) and the microtest wells are incubated at 18-25° C. for 74 min. Analysis is done at 405 nm. A standard curve is plotted each time the assay is performed. Various dilutions of each sample is analyzed. The test is done in the presence and absence of fibrin to compare the activity of CT t-PA in these two conditions.

According to one embodiment herein, the chimeric truncated tissue plasminogen activator t-PA protein is performed by utilizing standard protocols. Various concentrations of fibrinogen (0-0.3 mg/ml) are mixed with bovine thrombin (0.5 U/ml) in a buffer (0.05M Tris-HCl, pH 7.4, 0.12M NaCl, 0.01% Tween 80 and 1 mg/ml BSA) to for fibrin clots. After incubating the mixture at 37° C. for 30 min, and the clot is removed by centrifugation (15 min, 13000 rpm, 4° C.). The amount of enzyme bound to fibrin is calculated from the difference of the total amount of enzyme and the free enzyme in the supernatant as determined by Trinilize t-PA activity kit. The absorbance (405 nm) is measured after 20, 40 and 60 min.

According to one embodiment herein, the chimeric truncated tissue plasminogen activator t-PA protein is analyzed by the resistance assay of t-PA to inhibition by PAI-1. The human PAI-1 in different concentrations from 0 to 100 μg/ml is incubated with commercial full-length t-PA and CT t-PA (in 3000 IU/ml final concentration) at 25° C. Further the residual activity is measured after 1 hour of incubation. The residual activity is determined using the quantitative ELISA based Trinilize t-PA Activity kit.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flowchart indicating a method for synthesizing and testing the chimeric truncated tissue plasminogen activator (CT tPA), according to an embodiment herein.

FIG. 2 illustrates a graph indicating a Zeocin™ resisatant clones expression analysis, according to an embodiment herein.

FIGS. 3A and 3B illustrate graphs indicating Coomasie blue stained SDS-PAGE analysis from purified CT tPA and Western blot analysis of purified CT tPA, according to an embodiment herein.

FIG. 4 illustrates a graph indicating fibrin binding assay for analyzing affinity of CT tPA and Actylase®, according to an embodiment herein.

FIG. 5 illustrates a graph indicating the residual activity of Actylase® and PAI-1 resistant CT tPA after inhibition by rPAI-1, according to one embodiment herein.

FIG. 6 illustrates a schematic representation of chimeric truncated tissue plasminogen activator (CT tPA), according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a novel chimeric truncated form of tissue plasminogen activator (t-PA). The chimeric truncated form of tissue plasminogen activator (t-PA) is expressed in Pichia pastoris. The novel tissue plasminogen activator (t-PA) comprises of a finger domain of Desmoteplase, an epidermal growth factor (EGF) domain, a kringle 1 (K1) domain, a kringle 2 (K2) domain and a protease domain. In kringle 2 (K2) domain the lysine binding site (LBS) is deleted. In protease domain the four amino acids lysine 296, arginine 298, arginine 299 and arginine 304 are substituted by aspartic acid (DDDD). The chimeric truncated form of tissue plasminogen activator (t-PA) show increased activity in the presence of fibrin. Further chimeric truncated form of tissue plasminogen activator (t-PA) shows greater affinity towards fibrin than commercially available full length t-PA (Actylase®).

According to one embodiment herein, a chimeric truncated tissue plasminogen activator CT t-PA comprises of a native human t-PA with a F domain, an EGF domain, a K1 domain, a K2 domain and a protease (P) domain. The F domain of native human t-PA is replaced by F domain of vampire bat plasminogen activator. The 24 amino acids (LBS) of the K2 domain are deleted at a position of 202-225. The amino acids K296, R298, R299 and R304 in the P domain are replaced by four aspartic acids (DDDD).

According to one embodiment herein, the chimeric truncated t-PA has 503 amino acids. The chimeric truncated t-PA has a molecular weight of 65 kDa. The chimeric truncated t-PA has a 3N-glycosylation at residues N117, N184 and N448. The chimeric truncated t-PA has a specific activity of 5,200 IU/μg. The chimeric truncated tPA is resistance to a plasminogen activator inhibitor-1 (PAI-1) and the CT t-PA retains more than 90% of its biological activity in the presence of a fibrinogen. The chimeric truncated t-PA has a residual activity of 91%. Also the chimeric truncated t-PA has an amodolytic activity of 1,860 IU/ml in the absence of fibrin. The chimeric truncated t-PA has an amidolytic activity of 2,500,000 IU/ml in the presence of fibrin.

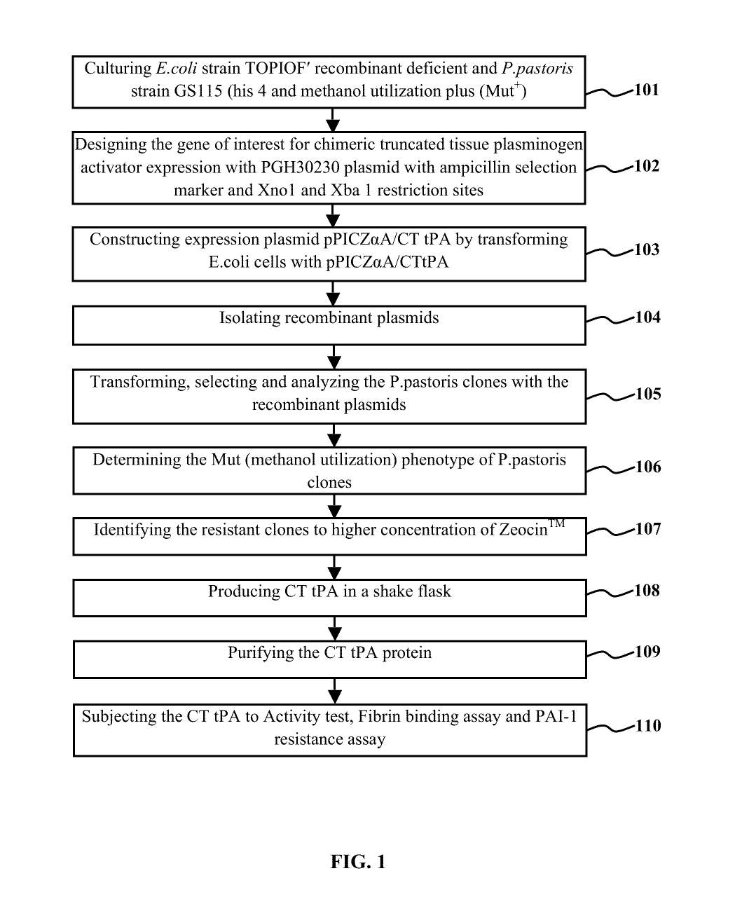

FIG. 1 illustrates a flowchart indicating a method for synthesizing and testing the chimeric truncated tissue plasminogen activator (CT tPA), according to an embodiment herein. With respect to FIG. 1, the first step is culturing E. coli strain TOP10F′ and P. pastoris strain GS115 (his 4 and methanol utilization plus (Mut⁺) (101). Further designing the gene of interest for chimeric truncated tissue plasminogen activator expression with pGH 30230 plasmid with ampicillin selection marker and Xno1 and Xba1 restriction sites (102). Next step is constructing expression plasmid pPICZαA/CT tPA by transforming E. coli cells with pPICZaA/CT tPA (103). The next step is isolating recombinant plasmids from transformed E. coli cells (104). Transforming, selecting and analyzing the P. pastoris clones with the recombinant plasmids (105). After analyzing the clones the next step is determining of Mut (methanol utilization) phenotype of P. pastoris clones (106). Identifying the resistant clones to higher concentration of Zeocin™ (107). Producing CT tPA in a shake flask (108). Purifying the CT tPA protein (109). Subjecting CT tPA to Activity test, Fibrin binding assay and PAI-1 Restriction assay (110).

### EXPERIMENTAL DETAILS

### Materials and Methods

Strains, Plasmids, Culture Medium, and Reagents:

The E. coli strain TOP10F′ which is recombination deficient (recA) and deficient in endonuclease A was used for all DNA manipulations. The P. pastoris strain GS115 (his4 and methanol utilization plus (Mut⁺)) and pPICZaA (Invitrogen) were kindly provided by Dr. Keyvan Madjidzadeh (Pasteur Institute of Iran).

TOP10F cells were cultured in Luria-Bertani medium (LB medium; 1% (w/v) tryptone, 0.5% (w/v) yeast extract, and 1% (w/v) NaCl, pH 7.0). Antibiotics were added to LB medium at the following final concentrations: 100 μg/ml ampicillin and 25 μg/ml Zeocin™. Low-salt LB medium (1% (w/v) tryptone, 0.5% (w/v) yeast extract, and 0.5% (w/v) NaCl, pH 7.5) was used during Zeocin™ selection procedure.

The P. pastoris strain GS115 was cultured in yeast extract peptone dextrose medium (YPD medium). The YPD medium comprises of 1% (w/v) yeast extract, 2% (w/v) peptone, and 2% (w/v) dextrose), for YPDS the YPD was supplemented with 1 M sorbitol. Buffered glycerol complex medium (BMGY) the buffered glycerol complex medium comprises of 1% (w/v) yeast extract, 2% (w/v) peptone, 100 mM potassium phosphate pH 6.0, 1.34% (w/v) yeast nitrogen base, 4×10⁻⁵% (w/v) biotin, 1% (v/v) glycerol) and buffered methanol complex medium (BMMY) in which the glycerol in BMGY was replaced with 0.5% (v/v) methanol. For plates, agar was added to a final concentration of 1.5% (w/v). Cultivation of P. pastoris strains is done at 30° C. For P. pastoris, Zeocin™ was added to a final concentration of 100 μg/ml for selection of transformants.

Restriction enzymes, T4 DNA ligase, DNA markers, and protein markers were purchased from Fermentas®. The primary polyclonal rabbit anti-tPA antibody was purchased from Abcam and the secondary antibody, peroxidase conjugated goat antirabbit antibody was obtained from Santa Cruz.

Design of the Gene of Interest:

To increase fibrin affinity and specificity, the bPA finger domain was replaced by the human tPA finger domain and 24 amino acids in the K2 domain, known as LBS, were deleted. In addition, for half-life prolongation, amino acids K296, R298, R299, and R304 were replaced by four aspartic acids (DDDD) in the protease domain, responsible for the resistance to the PAI-1. Thus, the nucleotide sequence of the novel CT t-PA gene encodes a protein of 503 amino acids with a molecular weight of 65 kDa and has 3 N-glycosylation sites at residues N117, N184, and N448. The CT t-PA contains a Desmoteplase finger domain (f(vamp)) followed by a full length human t-PA EGF domain and K1 domain. Downstream of these domains are the human t-PA K2 domain with the LBS deletion (D 202-225) and the protease domain with four aspartic acids substitutions (DDDD) at residues 296, 298, 299, and 304.

Construction of the Expression Plasmid pPICZaA/CT t-PA:

The gene coding for the new CT t-PA was synthesized in pGH-30230 plasmid and had an ampicillin selection marker as well as Xho1 and Xba1 restriction sites flanking the gene.

The vector for the production of CT t-PA in P. pastoris was constructed using the pPICZaA vector as backbone. The final vector provided the alpha mating factor from S. cerevisiae at the 50 end of the target gene to allow secretion as well a His6-tag at the 30 end for simple downstream process. The plasmid (pPICZaA/CT t-PA) was transformed into E. coli and selected on LB plates containing 25 μg/ml Zeocin™. Transformants were selected and verified by PCR, sequencing and digestion analysis. One positive transformant was grown in 100 ml liquid LB containing Zeocin™ (25 μg/ml) for 12 h and the recombinant plasmid (pPICZaA/CT t-PA) was isolated using a QIA quick column (Mini-Prep Kit, Qiagen) and sequenced.

Transformation, Selection, and Analysis of P. pastoris Clones:

About 10-20 μg of the recombinant plasmid were linearized using Sac 1 (Fermentas®) and were transformed into P. pastoris GS115 by electroporation using a MicroPulser (Bio-Rad). The parent construct pPICZaA, which lacked an insert, was used as negative control. After transformation, cells were spread on YPD plates containing Zeocin™ (0.1 mg/ml) and incubated at 30° C. for 3 days. Large colonies were selected and the integration of the CT t-PA gene into the genome was confirmed by direct colony PCR using 5′ AOX1 and 3′ AOX1 primers. The PCR reactions contained 5 μl of 10×PCR buffer, 2.5 μl of 50 mM MgCl₂ 2.5 μl of 10 mM dNTP, and 50 pmol of each primer (final volume 25 μl) and was carried out for 30 cycles. The PCR reaction was incubated at 95° C. for 5 min before adding the 2 μl of Taq DNA polymerase. Each cycle consisted of 1 min at 95° C., 30 s at 65° C., and 1 min at 72° C., with a final extension step of 5 min at 72° C.

Determination of Mut (Methanol Utilization) Phenotype of the Clones:

The methanol utilization phenotype was determined as described in the Invitrogen Instruction Manual (www.invitrogen.com/content/sfs/manuals/easyselect_man.pdf). Briefly, Zeo® transformants were streaked first onto minimal methanol (MM) and then on minimal dextrose (MD) agar plates in a matching regular pattern and were incubated at 30° C. for 48 h. The composition of the MD plate was similar to that of the MM plate, except that methanol was replaced by 2% (w/v) dextrose. To differentiate methanol utilization plus (Mut⁺) from methanol utilization slow (Mut⁺) phenotype, GS115/His⁺Mut^{S} Albumin and GS115/His⁺Mut⁺ β-gal strains were included as Mut^{s} and Mut⁺ controls in both MM and MD plates, respectively. After 2 days of incubation at 30° C., the clones were scored for growth with reference to the controls. Those that grew similar to the Mut⁺ control in the presence of methanol were selected as Mut⁺ Clones and used for further analysis.

Identification of Resistant Clones to Higher Concentration of Zeocin™:

It is known that clones which are resistant to a higher concentration of Zeocin™ might harbor multiple copies of the target gene. Therefore, we screened the transformants for potential multi-copy integration of CT tPA using a Zeocin™ screening procedure: Zeocin™ resistant clones were streaked on YPD plates containing increasing concentrations of Zeocin™ (0.2, 0.8, and 1.6 mg/ml of Zeocin™). After 4 days of incubation at 30° C., clones were evaluated for their ability to grow in the presence of rising concentrations of the antibiotic. Clones which were able to grow on YPD plates containing 1.6 mg/ml Zeocin™ were selected and finally stored as glycerol stocks at −80° C.

CT t-PA Production in Shake Flask:

Glycerol stocks were refreshed on YPD agar plates containing 0.1 mg/ml Zeocin™. Then, the cells were cultivated in 100 ml fresh BMGY medium in a 250 ml baffled flask at 30° C. and 250 rpm over night. After that, the culture was harvested by centrifugation at 1,500 g at room temperature for 5 min and the collected pellet was resuspended in 200 ml of BMMY in a 1 liter baffled flask. Induction was performed for 5 days at 25° C. at 250 rpm. Methanol (1% v/v) was added every 24 h to maintain induction. Samples were taken at 24 h intervals, spun down, and the clarified supernatants were diluted 20-fold in PBS, pH 7.6, and used for further analysis.

Protein Purification:

CT t-PA protein was purified from the culture medium with fast protein liquid chromatography (FPLC) using Ni-NTA agarose (Qiagen). Approximately a 15 ml Ni-NTA gel bed in a XK 16/20 column (Pharmacia) was equilibrated with 10 column volumes (CV) of 50 mM sodium phosphate pH 8.0, 300 mM NaCl, 10 mM imidazole. Cell-free cultivation broth was dialyzed in PBS buffer using a 25 kDa cut off Spectra/Por® membrane tube (Spectrum labs) at 4° C. over night. Then, the sample was loaded onto the Ni-NTA resin. Bound proteins were washed using 50 mM sodium phosphate pH 8.0, 0.3 M NaCl, and 20 mM imidazole (5 CV) and eluted with 50 mM sodium phosphate pH 8.0, 0.3 M NaCl, 300 mM imidazole into eight fractions which were then analyzed by SDS-PAGE.

Total Protein Determination:

The protein content of the samples was determined using the Bradford method with the Quick Start™ Bradford Dye Reagent. Bovine serum albumin (BSA) was used as standard in concentrations of 0.1-1.0 mg/ml.

SDS-PAGE and Western Blot:

SDS-PAGE was carried out using a 12% resolving polyacrylamide gel and stained with Coomassie Blue R-250. For Western blot analysis, samples were transferred to a nitrocellulose membrane using a semi-dry electroblotting apparatus (Bio-Rad) at 18 V for 45 min in Towbin transfer buffer (25 mM Tris, 192 mM glycine) according to the manufacturers' instructions (Invitrogen™). The nitrocellulose membrane was washed three times with 50 mM Tris-HCl pH 7.4, 150 mM NaCl, and 0.1% (v/v) Tween-20 (TBST) and blocked under shaking at room temperature with 5% (w/v) non-fat dry milk diluted in TBST for 60 min. Then, the membrane was washed once with 19 PBS, pH 7.4, and 0.2% Tween 20. After washing, the nitrocellulose membrane was first incubated for 60 min at room temperature with a primary polyclonal rabbit anti-tPA antibody (Abcam) in a 1/1,000 dilution. Then, the membrane was washed three times with TBST before it was incubated with the secondary peroxidase conjugated goat anti rabbit antibody (Santa Cruz) in a 1/1,500 dilution. Finally, the nitrocellulose membrane was again washed and developed with di-aminobenzidine according to manufacturer's recommendations (Thermo Scientific). Cell-free cultivation broth of a strain carrying the pPICZaA vector only was included as negative control and commercial t-PA (Actylase®) was used as positive control.

Activity Test:

The activity test was done using the Trinilize t-PA Activity kit (Tcoag) which is a bio-functional immunosorbent assay (BIA) to quantify the activity of human t-PA. 100 μl of t-PA standards and culture broth supernatant were added to the micro test strip wells and were incubated on a microtest plate shaker at ambient temperature (18-25° C.) at 600 rpm for 20 min. The samples were captured by SP-322 monoclonal antibody on the microtest wells (pH 5.9) without inhibiting t-PA activity. After discarding the test samples, mild detergents were used to wash the wells. The t-PA substrate (plasminogen, a plasmin-sensitive chromogenic substrate, and t-PA activity promoters) was added in HEPES buffer (pH 8.5) and the microtest wells were incubated at 18-25° C. for 75 min. Analysis was done at 405 nm. A standard curve was done each time an assay was performed. Various dilutions of each sample were analyzed. This test was done in the presence and absence of fibrin to compare the activity of CT t-PA in these two conditions.

Fibrin Binding Assay:

The assessment of t-PA and CT t-PA binding to fibrin was done by previously reported methods. In brief, various concentrations of fibrinogen (0-0.3 mg/ml) were mixed with bovine thrombin (0.5 U/ml) in a buffer (0.05 M Tris-HCl, pH 7.4, 0.12 M NaCl, 0.01% Tween 80, and 1 mg/ml BSA) to form fibrin clots. After incubating the mixture at 37° C. for 30 min, equal units (3,000 units) of CT t-PA or commercial full-length t-PA were added. The mixture was incubated at 37° C. for 30 min, and the clot was removed by centrifugation (15 min, 13,000 rpm, 4° C.). The amount of enzyme bound to fibrin was calculated from the difference of the total amount of enzyme and the free enzyme in the supernatant, as determined by Trinilize t-PA Activity kit. The absorbance (405 nm) was measured after 20, 40, and 60 min.

PAI-1 Resistance Assay:

Resistance of t-PA to inhibition by PAI-1 was assessed by previously reported methods. Human rPAI-1 in different concentrations from 0 to 100 μg/ml were incubated with commercial full-length t-PA and CT t-PA (in 3,000 IU/ml final concentration) at 25° C. and residual activity was measured after 1 h. The residual activity was determined using the quantitative ELISA based Trinilize t-PA Activity kit.

### Results

Mut Phenotype Analysis and Selection of clones Resistant to High Level Zeocin™:

Mut phenotype analysis illustrates that all clones have a Mut⁺ phenotype. The clones which are resistant to higher Zeocin™ concentrations have a higher copy number of gene integration. Most of the transformants appeared on plates with 200 μg/ml Zeocin™ after 3-4 days of incubation, whereas only 12 transformants appeared on plates with 800 μg/ml Zeocin™. Further only six transformants could resist 1,600 μg/ml Zeocin™ after 5 days of incubation. The six transformants are taken for further analysis.

Expression and Purification:

The expression levels of the six different resistant clones to higher concentration of Zeocin™ are analyzed revealing that clone number 10 had the highest level of expression, namely 1,797 IU/ml, after 5 days of cultivation, whereas P. pastoris containing only a control expression plasmid (pPICZaA) had no such activity throughout the induction period.

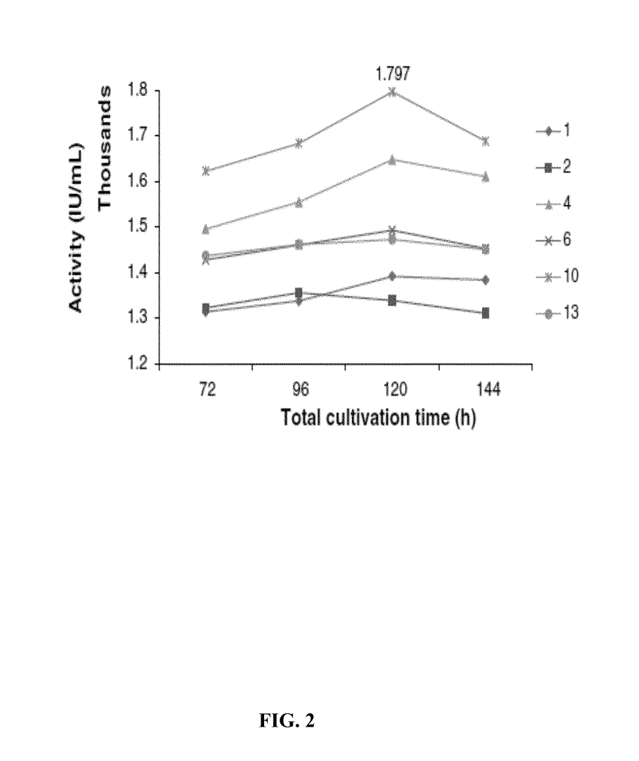

FIG. 2 illustrates a graph indicating a Zeocin™ resistant clones expression analysis, according to an embodiment herein. With respect to FIG. 2 the maximum production of all clones is reached after 5 days. The dilution factor (1/1000) of the sample is considered. The highest CT tPA activity is calculated 1,797 IU/ml on day 5 of the culture for clone 10.

SDS-PAGE and Western Blot:

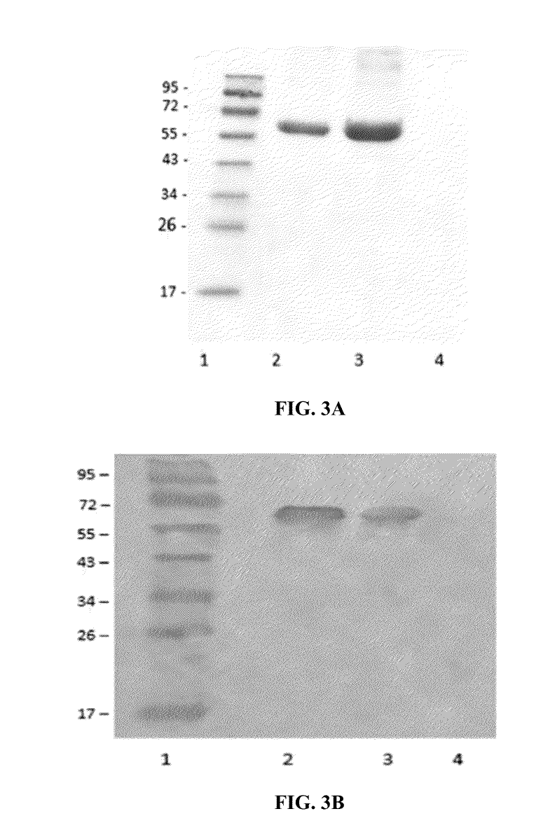

In subsequent protein purification using immobilized metal affinity chromatography the eluted protein emerged as a single sharp peak (graph not shown). The peak fractions are pooled and analyzed by SDS-PAGE and Western blot. FIGS. 3A and 3B illustrate graphs indicating Coomasie blue stained SDS-PAGE analysis from purified CT tPA and Western blot analysis of purified CT tPA, according to an embodiment herein. With respect to FIG. 3A, the figure illustrates SDS PAGE analysis after coomasie blue staining from purified CT tPA. The purity of the final purified CT tPA is more than 90%. FIG. 3A further illustrates lane 1 of gel comprising PageRuler™ prestained protein ladder (Fermentas®), lane 2 of the gel comprising 3 μg of commercial full-lengths tPA (Actylase®, 65 kDa), lane 3 of the gel comprises 5 μg of purified CT tPA (63 kDa) and lane 4 of the gel comprises negative control. FIG. 3A also illustrates that the purified CT tPA expressed in P. pastoris (63 kDa) and the commercial tPA (Actylase®, 65 kDa) appeared between 55 and 72 kDa.

With respect to FIG. 3B the figure illustrates the Western blot analysis of the purified CT t-PA. The lane 1 of the gel comprises PageRuler™ prestained protein ladder (Fermentas®), lane 2 of the gel comprises CT t-PA (63 kDa), lane 3 of the gel comprises commercial full-length tPA (Actylase®, 65 kDa) and the lane 4 of the gel comprises negative control. The FIG. 3B also illustrates the Western blot bands at the appropriate size for the CT t-PA expressed in P. pastoris. The Bradford protein assay further shows that approximately 100 mg of CT t-PA protein is obtained from 200 ml of culture supernatant.

Biological Activity of CT t-PA in the Presence of Soluble Fibrin:

The amidolytic unit of the purified sample is determined to be about 2,500,000 IU/ml in the presence of 50 μg of soluble fibrin, whereas the amount for this clone in the absence of fibrin is only 1,800 IU/ml. Further CT t-PA exhibits an over 1,300-fold higher activity in the presence of fibrin compared to a condition in which fibrin was absent, while this ratio for the commercial full-length t-PA (Actylase®) was only about 700-fold. Another remarkable point was the potency of CT t-PA, which was about 5,000,000 IU/mg. This potency is 10-fold higher than for the commercial full-length t-PA Actylase®, which is reported with 570,000 unit/mg. Further analysis of CT t-PA reveals that the amidolytic activity is 1,860 IU/ml in the absence of fibrin, while the activity increases more than 13 fold and reached 2,500,000 IU/ml in the presence of fibrin.

Fibrin Binding Assay:

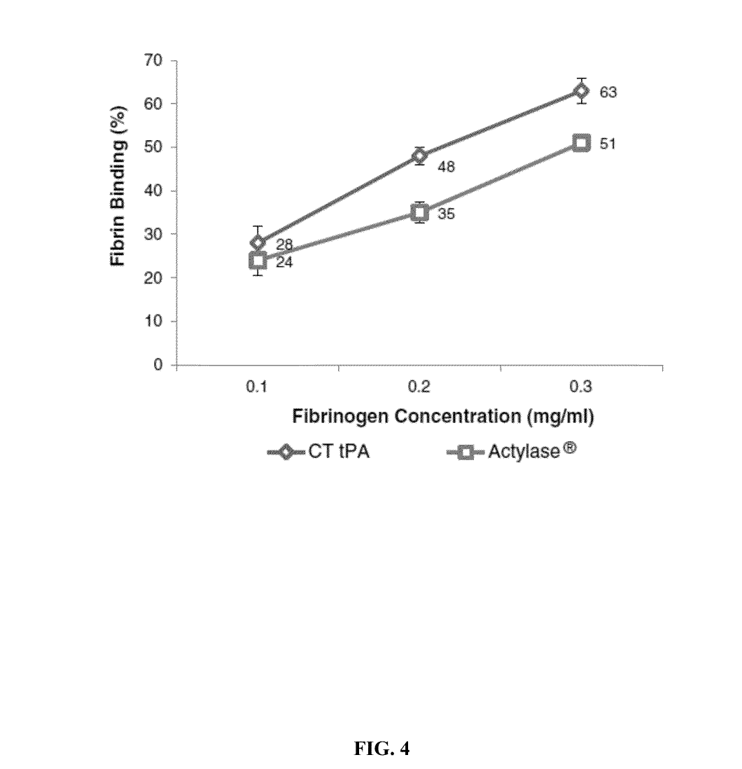

FIG. 4 illustrates a graph indicating fibrin binding assay for analyzing affinity of CT tPA and Actylase®, according to an embodiment herein. FIG. 4 further illustrates 48% of CT t-PA successfully bound to fibrin in 0.2 mg/ml concentration of fibrinogen while this amount for Actylase® is only 35%. In 0.3 mg/ml concentration of fibrinogen, CT t-PA shows 63% binding to fibrin whereas this value for Actylase® is only 51%. According to these results, CT t-PA showed a 1.2-fold higher affinity toward fibrin protein compared to Actylase®. Also the specific activity of the CT tPA is 5200 IU/μg.

PAI-1 Resistance Assay:

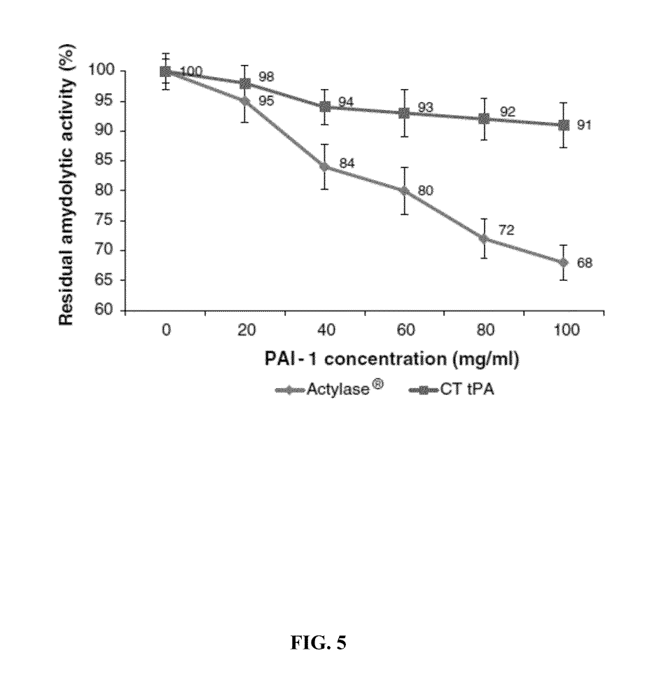

FIG. 5 illustrates a graph indicating the residual activity of Actylase® and PAI-1 resistant CT t-PA after inhibition by rPAI-1, according to one embodiment herein. The level of resistance of CT t-PA and Actylase® towards the human rPAI-1 molecule is determined in vitro. As shown in FIG. 5, after 1 h of incubation of CT t-PA with rPAI-1 (100 μg/ml), only 9% of protein activity is neutralized, whereas 32% of Actylase® activity is lost. Further analysis illustrates that in the presence of PAI-1, Actylase® only retained 68% of its activity whereas CT t-PA retains more than 90% of its activity. CT t-PA activity indicates that CT t-PA has a higher resistance towards the inhibitor. The residual activity of CT t-PA is 91% when incubated with PAI-1.

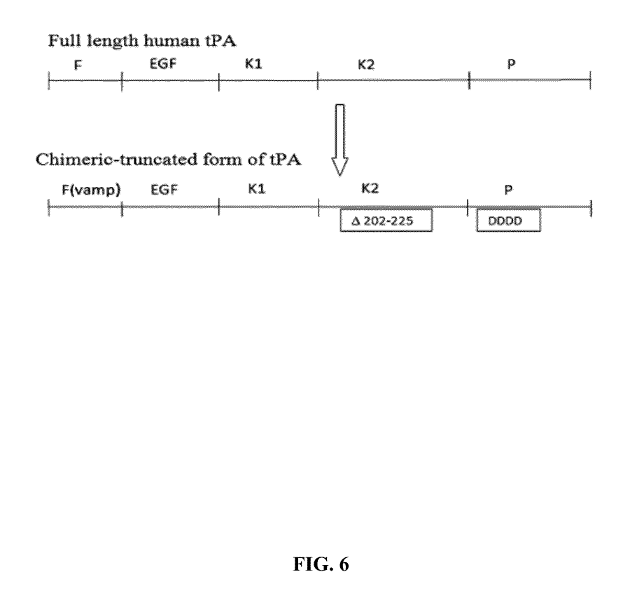

FIG. 6 illustrates a schematic representation of chimeric truncated tissue plasminogen activator (CT t-PA), according to one embodiment herein. To increase fibrin affinity and specificity the vampire bat plasminogen activator (bPA) finger domain is replaced by the human t-PA finger domain and 24 amino acids in the K2 domain (LBS) are deleted. Further for the half life prolongation amino acids K296, R298, R299 and R304 are replaced by four aspartic acids (DDDD) in the protease domain, responsible for the resistance to the PAI-1. The nucleotide sequence of the novel CT t-PA gene encodes a protein of 503 amino acids with a molecular weight of 65 kDa and has 3 N-glycosylation sites at residues N117, N184 and N448. FIG. 6 further illustrates that CT t-PA further comprises a Desmoteplase finger domain (f (vamp)) followed by a full-length human t-PA EGF domain and K1 domain. Downstream of these domains are the human t-PA K2 domain with the LBS deletion (Δ202-225) and the protease domain with four aspartic acids substitutions (DDDD) at residues 296, 298, 299 and 304.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A chimeric truncated tissue plasminogen activator CT t-PA comprising:
a native human t-PA with a F domain, an EGF domain, a K1 domain, a K2 domain and a protease (P) domain;
wherein the F domain of native human t-PA is replaced by F domain of vampire bat plasminogen activator, and wherein 24 amino acids (LBS) of the K2 domain are deleted at a position of 202-225, and wherein the amino acids K296, R298, 8299 and R304 in the P domain are replaced by four aspartic acids (DDDD).

2. The chimeric truncated t-PA according to claim 1, wherein the t-PA has 503 amino acids.

3. The chimeric truncated t-PA according to claim 1, wherein the t-PA has a molecular weight of 65 kDa.

4. The chimeric truncated t-PA according to claim 1, wherein the t-PA has a 3N-glycosylation at residues N117, N184 and N448.

5. The chimeric truncated t-PA according to claim 1, wherein the t-PA has a specific activity of 5,200 IU/μg.

6. The chimeric truncated t-PA according to claim 1, wherein the t-PA is resistance to a plasminogen activator inhibitor-1 (PAI-1) and wherein the CT tPA retains more than 90% of its biological activity in the presence of a fibrinogen.

7. The chimeric truncated t-PA according to claim 1, wherein the t-PA has a residual activity of 91%.

8. The chimeric truncated t-PA according to claim 1, wherein the t-PA has an amodolytic activity of 1,860 IU/ml in the absence of fibrin.

9. The chimeric truncated t-PA according to claim 1, wherein the t-PA has an amidolytic activity of 2,500,000 IU/ml in the presence of fibrin.
